(19) United States
(12) Patent Application Publication
(10) Pub. No.: US 2006/0179492 A1
(43) Pub. Date: Aug. 10, 2006

(54) Method of inhibiting neural transmission mediated by serotonin-2a and enhancing sensorimotor gating

(75) Inventor: David Feifel, La Jolla, CA (US)

Correspondence Address: BUCHANAN INGERSOLL LLP;(INCLUDING BURNS, DOANE, SWECKER & MATHIS), P.O. BOX 1404, ALEXANDRIA, VA, 22313-1404, US

(73) Assignee: The Regents of the University of California, Oakland, CA (US)

(21) Appl. No.: 10/538,245

(22) PCT Filed: Dec. 8, 2003
(86) PCT No.: PCT/US03/39196

§ 371 Date: Jun. 7, 2005

Related U.S. Application Data
(63) Continuation-in-part of application No. 60/431,937, filed on Dec. 9, 2002.

(51) Int. Cl. A61K 49/00; A01K 67/027

(52) U.S. Cl. 800/3; 424/9.2

(57) ABSTRACT

Disclosed are methods for treating neuropsychiatric disorders and disorders associated with sensorimotor gating deficits and/or prepulse inhibition disorders and improving sensorimotor gating in normal subjects. Also provided are animal models useful for identifying agents that modulate sensorimotor gating activity for preclinical testing of psychotropic drugs.

### CROSS REFERENCE TO RELATED APPLICATIONS

The invention claims priority under 35 U.S.C. §119 to provisional application Ser. No. 60/431,937, filed Dec. 9, 2002, the disclosure of which is incorporated herein by reference.

### STATEMENT REGARDING FEDERALLY SPONSORED RESEARCH

The invention was funded in part by Grant No. MH6245-01 awarded by the National Institutes of Health and by Grant No. 955962 awarded by the National Alliance For Research on Schizophrenia and Depression. The government may have certain rights in the invention.

### TECHNICAL FIELD

This invention relates to methods and compositions useful in screening for agents that treat neurological disorders and methods and compositions for treating neurological disorders.

### BACKGROUND

Neurotensin (NT) is a neuropeptide that is co-localized with dopamine. Centrally administered NT inhibits dopamine function. Therefore, investigators have suggested that NT agonists may have potential as antipsychotic drugs (Nemeroff et al., Ann. N.Y. Acad. Sci. 668:147-56, 1992). The short half-life and inability to cross the blood-brain barrier have limited the development of neuropeptides, such as NT, as psychotropic drugs.

Sensorimotor gating refers to the central nervous system's ability to modulate and filter incoming environmental information. It is thought that disruption of sensorimotor gating gives way to cognitive flooding in which environmental information overwhelms the central nervous system. It is believed that psychiatric symptoms such as psychosis, confusion or impaired cognitive functioning are the result of improper sensorimotor gating. Therefore, improving sensorimotor gating among subjects who suffer from a neuropsychiatric disorder associated with sensorimotor gating abnormalities may reduce their symptoms. Since sensorimotor gating exists on a continuum, it is also possible that enhancing sensorimotor gating in normal people may enhance their cognitive abilities (i.e., their ability to focus, sustain attention).

The invention provides neurotensin agonists that are useful as agents that inhibit neural processes produced by activation of dopamine-2, sertonin-2A and alpha-1 noradrenergic (sometimes referred to as adrenergic) receptors. Such neurotensin agonists have efficacy for the treatment of neuropsychiatric disorders, including, but not limited to, schizophrenia, schizoaffective disorder, mania, bipolar affective disorder, depression, anxiety, obsessive-compulsive disorder, post-traumatic stress disorder, autism, and agitation. Neurotensin agonists enhance prepulse inhibition (PPI), a measure of sensorimotor gating, in subjects having PPI deficits thereby modulating sensorimotor gating. PPI deficits are common in a number of neuropsychiatric disorders. Neurotensin agonists are effective in treating such disorders because of an agonist's ability to have an effect on reversing perturbation produced in multiple components of the neurological circuitry underlying sensorimotor gating, rather than reversing a specific part of the circuitry as do most antipsychotics. In addition, neurotensin agonists enhance normal sensorimotor gating and therefore enhance information processing and cognition above baseline in normal subjects.

The disclosure provides a method for identifying an agent useful for treating neuropychiatric disorders. The method includes (a) administering an agent to an animal model exhibiting inherent reduced prepulse inhibition (PPI), (b) subjecting the animal model to a startle stimulus, and (c) observing the magnitude of a PPI in the animal model, wherein a increase in PPI compared to a control animal is indicative of an agent that is useful for treating neurological disorders. In one embodiment, the animal model is a Brattleboro Rat model or a Brown Norway Rat model.

Also disclosed are methods for screening a test psychotropic agent for treating a neuropsychiatric disorder. The methods include (a) administering to an animal a test psychotropic agent; and (b) measuring prepulse inhibition in a startle reflex response chamber, wherein an increased prepulse inhibition level indicates a strong clinical potential as a psychotropic drug. The animal can be a Brown Norway Rat or a Brattleboro Rat.

The disclosure provides an animal model for obtaining preclinical predictive information concerning effectiveness of a treatment for neuropsychiatric disorders. The animal model has inherently reduced prepulse inhibition compared to normal or control animals due to genetic differences. In one aspect, the animal model is a Brattleboro rate or a Brown Norway rat. A method for obtaining predictive information about a treatment includes (a) administering to an animal a test psychotropic agent; and (b) measuring a prepulse inhibition in a startle reflex response chamber, wherein an increased prepulse inhibition indicates a strong clinical potential as a psychotropic drug.

The disclosure further provides a method of modulating sensorimotor gating in a subject comprising administering to a subject an agent selected from the group consisting of a neurotensin (NT), an NT analog, an NT agonist, and a combination thereof, in an amount effective to increase prepulse inhibition thereby modulating sensorimotor gating. In one aspect the agent is selected from the group consisting of neurotensin, any fragment thereof including neurotensin (from about residue x to residue 13 of SEQ ID NO:1-pGlu-Leu-Tyr-Glu-Asn-Lys-Pro-Arg-Arg-pro-Tyr-Ile-Leu-OH) wherein x is any number less than 11, or any modified version of neurotensin or a neurotensin fragment thereof, including but not limited to (Boc-Lys⁹)-neurotenin(9-13)-methyl ester, (Dab⁹)-neurotensin(8-13), (Dab⁹)-neurotensin(9-13), (Lys⁹, Trp¹¹, Glu¹²)-neurotensin(8-13), PD149163, NT1, NT2, NT64D, NT64L, NT65L, NT66D, NT66L, NT67L, NT69L, NT69L′, NT71, NT72, NT73, NT74, NT75, NT76, and NT77. Non-peptide compounds are included within the scope of the term “neurotensin agonists” including non-peptide drugs that are antagonists at certain neurotensin receptors and agonists at others including, but not limited to, levocobastine, SR48692, and SR142948. In addition, small molecule mimetics that can interact with neurotensin receptors are also encompassed within the scope of the disclosure. Such small molecule mimetics can be identified by using the animal models and assays described herein to identify or determine the efficacy of such molecules.

In another aspect, the disclosure provides methods of treating a neuropsychiatric disorder selected from the group consisting of depression, postpartum depression, affective disorder, schizoaffective disorder, schizophreniform disorder, delusional disorder, brief psychotic disorder, shared psychotic disorder, borderline personality disorder, manic-depressive disorder, obsessive-compulsive disorder, Huntington's Disease, Tourette's syndrome, bipolar affective disorder, autism, anxiety disorders, agitation and tic disorders, by administering to a subject an NT agonist.

The disclosure further provides a method for treating a subject having a neuropsychiatric disorder comprising administering to the subject a pharmaceutically effective dose of an NT agonist or a pharmaceutically acceptable salt thereof.

Also provided by the disclosure is a method of inhibiting serotonin-serotonin-2A and/or alpha-1 receptor mediated neural function comprising administering to a subject an effective amount of a neurotensin (NT) agonist.

In another aspect, the disclosure provides a method of improving cognitive function and/or memory attention in a subject comprising administering to a subject an agent selected from the group consisting of a neurotensin (NT), an NT analog, an NT agonist, and a combination thereof, in an amount effect to improve cognitive function and/or memory attention compared to a control subject.

The details of one or more embodiments of the invention are set forth in the accompanying figures and the description below. Other features, objects, and advantages of the invention will be apparent from the description and drawings, and from the claims.

### DESCRIPTION OF DRAWINGS

FIG. 1 illustrates prepulse inhibition of the startle reflex.

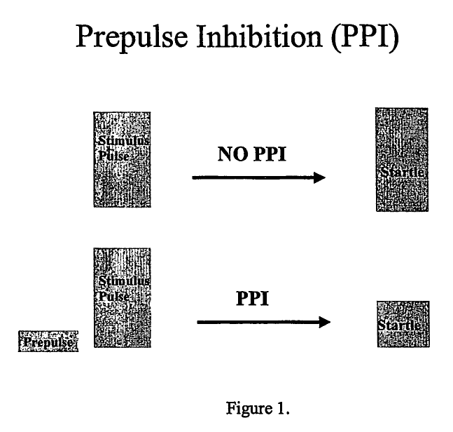

FIG. 2 shows the prepulse inhibition in normal subjects and schizophrenic subjects.

FIG. 3 is a representation of a possible mechanism of NT agonistic activity on sensorimotor circuits and symptoms of neuropsychiatric disorders.

FIG. 4 shows the effect of NT69L on PPI (Main) and startle magnitude (Inset) in rats receiving saline or DOI. Significant differences from the rats treated with DOI and the same dose of NT69L are represented by ** P<0.01. Significant differences from the rats receiving 0 mg/kg of NT69L and the same DOI treatment are represented by + P<0.05 and ++ P<0.01.

FIG. 5 is a plot showing the effect of PD149163 on PPI (Main) and startle magnitude (Inset) in rats receiving saline or cirazoline. Significant differences from the rats treated with cirazoline and the same dose of PD149163 are represented by ** P<0.01. Significant differences from the rats receiving 0 mg/kg (vehicle only) of PD149163 and the same cirazoline treatment are represented by +P<0.05 and ++P<0.01.

FIG. 6 is a plot showing the effect of NT69L on PPI (Main) and startle magnitude (Inset) in rats receiving saline or cirazoline. Significant differences from the rats treated with cirazoline and the same dose of NT69L are represented by * P<0.05 and ** P<0.01. Significant difference from the rats receiving 0 mg/kg (vehicle only) of NT69L and the same cirazoline treatment is represented by +P<0.05.

FIG. 7 shows prepulse inhibition (main) and acoustic startle response (inset) in rats treated with haloperidol. Significantly different than vehicle-treated LE rats represented by * (P<0.05) and ** (P<0.01). Significantly different than vehicle-treated BB rats represented by ## (P<0.01).

FIG. 8 is a plot that shows prepulse inhibition (main) and acoustic startle response (inset) in rats treated with clozapine. Significantly different than vehicle-treated LE rats represented by * (P<0.05) and ** (P<0.01). Significantly different than vehicle-treated BB rats represented by # (P<0.05).

FIG. 9 is a plot that depicts prepulse inhibition (main) and acoustic startle response (inset) in rats treated with PD149169. Significantly different than vehicle-treated LE rats represented by ** (P<0.01). Significantly different than vehicle-treated BB rats represented by # (P<0.05) and ## (P<0.01).

FIG. 10 is a plot depicts prepulse inhibition (main) and acoustic startle response (inset) in Brown Norway (BN) rats treated with saline, haloperidol, clozapine or PD149169. Significantly different than vehicle-treated BN rats represented by *(P<0.05).

### DETAILED DESCRIPTION

As used herein, an “agonist” is any compound that acts directly or indirectly through or upon a receptor to produce a pharmacological effect, while an “antagonist” is any compound that blocks the stimulation of a receptor and its resulting pharmacological effect. Enhancing PPI is a feature associated with some antipsychotics and is thought to be associated with their therapeutic mechanism. This occurs despite the fact that these neurotensin agonists do not directly interact with the same pharmacological systems as antipsychotics.

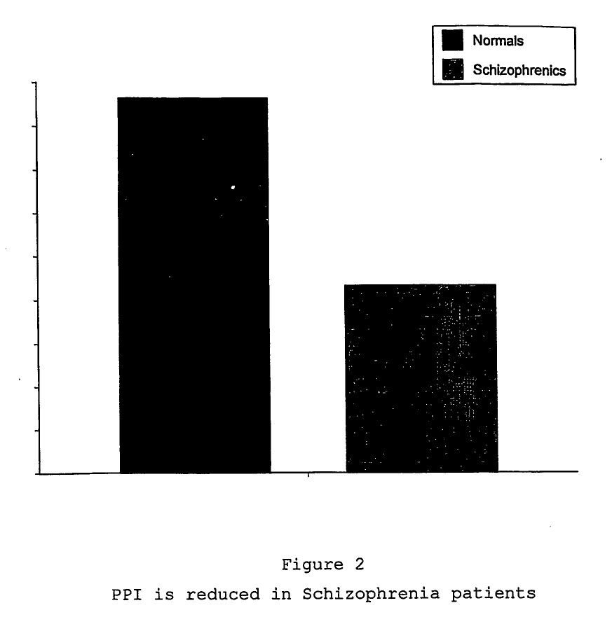

Sensorimotor gating refers to the central nervous system's ability to modulate and filter incoming environmental information. There are several ways of measuring sensorimotor gating, which is a common normal brain function among mammals, including humans. The most popular and widely studied method uses a phenomenon called prepulse inhibition (PPI) of the startle reflex as an operational measure of sensorimotor gating. PPI refers to the normal reduction in an animal or human of a startle response to a sudden stimulus when that stimulus is preceded very rapidly by a milder stimulus. PPI is reduced in a number of neuropsychiatric disorders, including Huntington's disorder, Schizophrenia, Tourette disorder, Obsessive-Compulsive Disorder, and Bipolar disorder (see, e.g., FIG. 2). This phenomenon is thought to reflect a disruption in cortico-striatal-pallidal-pontine circuits involved in pre-conscious processing of environmental stimuli (Geyer et al., Psychopharm. (BERL) 156(2):117-54, 2001). It is thought that disruption of sensorimotor gating gives way to cognitive flooding in which environmental information overwhelms the central nervous system. It is believed that psychiatric symptoms such as psychosis, confusion or impaired cognitive functioning are the result of improper sensorimotor gating. Therefore, improving sensorimotor gating among patients who suffer from a neuropsychiatric disorder associated with sensorimotor gating abnormalities may reduce their symptoms. Since sensorimotor gating exists on a continuum, it is also possible that enhancing sensorimotor gating in normal subjects may enhance their cognitive abilities (i.e., their ability to focus, sustain attention, and the like).

PPI deficits analogous to those seen in schizophrenia can be induced in rats by administering psychomimetic drugs of several different pharmacological families including dopamine agonists such as amphetamine and apomorphine (Mansbach et al., Psychopharm. 94:507-14, 1988), serotonin agonists such as DOI, and non-competitive NMDA antagonists such as phencyclidine (PCP) and dizocilpine (MK801) and the alpha-1 adrenergic agonist, cirazoline (Mansbach and Geyer, Neuropsychopharm. 2(4):299-308, 1989). Furthermore, established antipsychotics, can reverse the disruption of PPI produced by these drug to various degrees depending on their pharmacological profile, making PPI the basis of a predictive model for antipsychotic drugs (Geyer et al., supra). For example, PPI deficits produced by dopamine agonists can be reversed by all established antipsychotics since inhibition of dopamine-2 receptors is a common feature of all currently approved antipsychotics. However, only drugs which inhibit serotonin-2A receptor functioning block PPI disruption produced by the serotonin-2A agonist, DOI, a member of the hallucinogenic drug family. Thus, second generation or “atypical” antipsychotics, but not first generation or “typical” antipsychotics, reverse PPI disruption produced by DOI, since only the former are strong antagonists at the serotonin-2A receptor.

Thus drug-induced disruption of PPI in animals has been widely used as predictive model of antipsychotic activity, and is considered among the most useful predictive preclinical models of antipsychotic potential. However, the drug-induced PPI disruption model has significant limitations. PPI deficits in patients with neuropychiatric disorders mentioned above do not require drug intake or any other manipulation to produce such PPI/sensorimotor disorders but are rather inherent features of these patients, likely a result of genetic influences. Furthermore, as screening techniques for potential new antipsychotics, drug-induced models limit discovery to drugs that pharmacological oppose the specific drug used to disrupt PPI. Therefore, there is significant interest in development of non-pharmacological paradigms of PPI deficits. Two of the most studied examples of non-pharmacological paradigms of PPI deficits are social isolation-rearing and neonatal hippocampal lesions. However, both these models also require manipulations to produce PPI deficits similar to the deficits seen inherently in patients with schizophrenia and other neuropsychiatric disorders.

Brattleboro (BB) rats are Long Evans (LE) rats with a single base pair mutation that results in the inability to properly synthesize the neurotransmitter and neurohormone vasopressin. Brattleboro rats have many behavioral and cognitive abnormalities, including deficits in memory, emotion, social recognition, motivation and attention. They also have abnormalities in brain systems including dopamine and serotonin, neurotransmitters implicated in schizophrenia. Furthermore, untreated BB rats have deficits in PPI compared with their wild type, LE counterparts. This disclosure demonstrates that acute treatment with a wide range of doses of haloperidol, the protoypical first generation antipsychotic did not reverse the PPI deficits in BB rats or alter PPI in their wild type (WT) Long Evans control counterparts. In contrast, the disclosure demonstrates that acute treatment with 10 mg of clozapine, the prototype of the second generation (atypical) antipsychotic family significantly enhanced PPI in BB but not WT rats, eliminating the differences in PPI between these two strains.

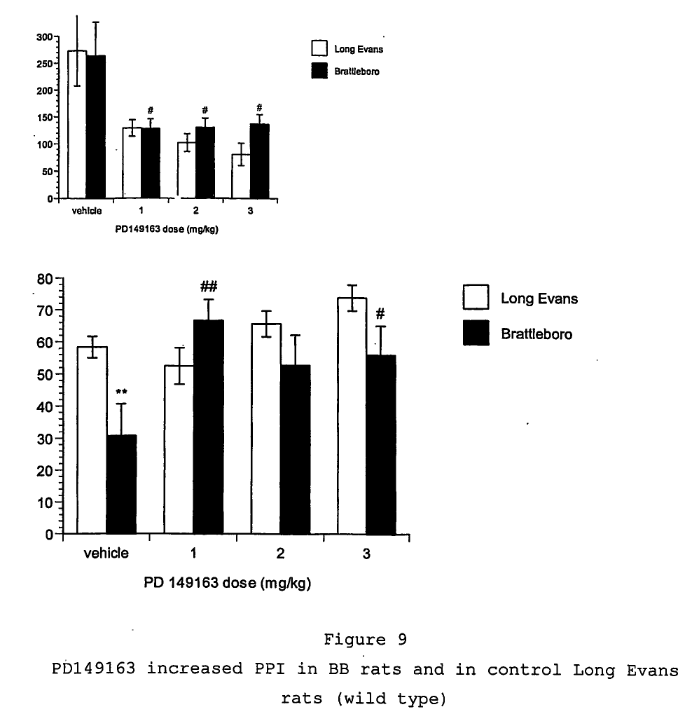

The fact that clozapine but not haloperidol reversed PPI deficits in BB rats suggests that acute reversal of PPI deficits in BB rats may also have utility as a predictive model for drugs that have properties of second generation antipsychotics, a family of medications that has been shown to be useful in treating the symptoms associated with a large number of neuropsychiatric disorders. This is supported by the finding that the putative antipsychotic PD149163, has a novel mechanism compared to established antipsychotics, being as an agonist at neurotensin receptors, significantly increased PPI in BB rat (see, e.g, FIG. 9).

Many patients with schizophrenia and other neuropsychiatric illnesses do not improve with currently available antipsychotic and are considered “treatment refractory.” Brown Norway (BN) is a strain of rats that have been shown to have low PPI. This disclosure demonstrates that acute administration of haloperidol or clozapine did not enhance PPI BN rats. In contrast, the putative antipsychotic PD149163, significantly increased PPI in BN rats through a novel mechanism compared to established antipsychotics. Thus, BN rats may be a useful model of “treatment refractory” schizophrenia and provide a useful predictive model to identify drugs that may be effective in patients who do not respond to first- or second-generation antipsychotics.

The BB and BN rats offer models of sensorimotor gating deficits and predictive models of antipsychotic potential with many novel and useful features. First, such models do not require pharmacological, environmental or neuroanatomical manipulations to produce PPI deficits. Rather, PPI deficits homologous to those seen in schizophrenia and other neuropsychiatric disorders are exhibited spontaneously and presumably due to the single gene abnormality in the BB rat and polygene strain differences in the case of the BN rats. As a genetic model of PPI deficits the BB and BN rat exhibits greater construct validity for the neuronsychiatric disorders associated with PPI deficits compared to models requiring pharmacological, environmental or neuroanatomical manipulations to produce PPI deficits. This improved construct validity affords the possibility of using the BB rats to explore the neurobiological and genetic substrates underlying sensorimotor gating abnormalities that may also underlie the neuropsychiatric conditions associated with such deficits. Because in the context of acute administration, the BB rat model appears to differentiate first generation from antipscyhotics not belonging to the first generation and/or drugs with clozapine-like efficacy from drugs with more conventional efficacy, it is a useful predictive screen for antipsychotic drugs with novel mechanism such as neurotensin agonists. Haloperidol's effect on BB PPI is greater after chronic administration than after acute administration. Therefore the BB rat model may also be useful for elucidating the mechanisms underlying the therapeutic time course, which is typically associated with antipsychotic treatment in schizophrenia patients. This disclosure provides a method for identifying an agent useful for treating psychiatric disorders. This method includes (a) administering an agent to an animal with inherently (e.g. genetically) low PPI, (b) subjecting the animal to startle stimulus according to PPI paradigm, (c) observing the level of PPI in the animal model, wherein an increase in PPI compared to a control animal is indicative of an agent that is useful for treating neuropsychiatric disorders. In one embodiment, the animal model is a Brattleboro Rat model or Brown Norway Rat model.

Haloperidol is a prototype of the first-generation, or “typical” family of antipsychotic drugs. These antipsychotics produce their therapeutic effects via a single pharmacological mechanism, inhibition of dopamine-2 receptor circuits which is produced because first generation antipsychotics bind to dopamine-2 receptors, and block effective binding of these receptors to dopamine (i.e. act as dopamine-2 antagonists).

First-generation antipsychotics have notable limitations with respect to clinical efficacy. For example, a significant proportion of schizophrenia patients fail to respond adequately to haloperidol and other antipsychotics from the first generation. Furthermore, experience with haloperidol and other first generation antipsychotics indicates that they have good efficacy against “positive” symptoms of schizophrenia (i.e., hallucinations, delusions) but they are less efficacious against “negative” symptoms (i.e., paucity of thought, decreased emotional expression, decreased volition behavior) or the cognitive deficits known to be associated with such neuropsychiatric disorders. As used herein, the term “neuropsychiatric disorder” is used interchangeably with the terms “psychosis,” “psychotic condition,” or analogous terms. A neuropsychiatric disorder includes, but is not limited to, psychotic depression, schizoaffective disorder, schizophreniform disorder, schizophrenia, delusional disorder, brief psychotic disorder, shared psychotic disorder, pychosis secondary to a general medical condition and psychosis—not otherwise specified.

More recently, a second-generation of antipsychotics has been developed. These “atypical” antipsychotics, of which clozapine is the prototype, appears to be more efficacious, particularly in ameliorating negative symptoms and cognitive deficits associated with schizophrenia. Furthermore, second generation antipsychotics produce fewer side effects associated with inhibition of dopamine circuits (e.g. abnormal movements, elevated prolactin levels) compared to first generation antipsychotics. The superior clinical efficacy and side effect profile associated with second-generation antipsychotics is due to their ability to bind and thereby inhibit function of serotonin-2A receptors in addition to dopamine-2 receptors. Other mechanism may contribute to the efficacy of second-generation antipsychotics, including inhibition of alpha-1 adrenergic receptor function.

While they were initially developed as treatments for neuropsychiatric disorders, second-generation antispychotics have proven useful in treating a wide array of non-psychotic neuropsychiatric illneses and symptoms. Accordingly, neuropsychiatric disorders also include bipolar affective disorder, mania, depressive disorders, obsessive-compulsive disorder, anxiety disorders, autism, personality disorders, impulse control disorders, tourettes disorder, huntingtons disorder, agitation, and aggression. The ability of second-generation antipsychotics to improve symptoms of these non-psychotic neuropsychiatric disorders, to an extent much greater than first generation antipsychotics, is due to their ability inhibit serotonin-2A receptors. Other mechanism may also contribute including inhibition of alpha-1 adrenergic receptors.

In addition to sharing clinical properties with all other atypical antipsychotics, clozapine is also considered by many clinicians and investigators to be singular among antipsychotics in regards to efficacy. This is particularly evident in the high success rates with clozapine among patients with schizophrenia who do not respond to other antipsychotics.

Neurotensin is a 13-amino acid natural peptide, which exists in the brain and in the periphery. Neurotensin receptors in exist throughout the brain and in peripheral organs. Neurotensin and neurotensin agonist produce several physiological effects, including regulation of temperature. The 8-13 amino acid fragment of neurotensin is one of the smallest fragments that retains full biological activity with respect to many of the physiological effects produced by the parent tridecapeptide. More recently, second-generation antipsychotics have been introduced into the marketplace and have progressively replaced first-generation antipsychotics as the standard of care treatment for psychotic disorders. Thus, the ability of neurotensin or neurotensin agonists to inhibit dopamine-2 receptor function, the sole mechanism underlying first generation antipsychotics, does not, by itself, support the notion that these compounds are good candidates to be developed as useful antipsychotic drugs based upon the contemporary standards of care.

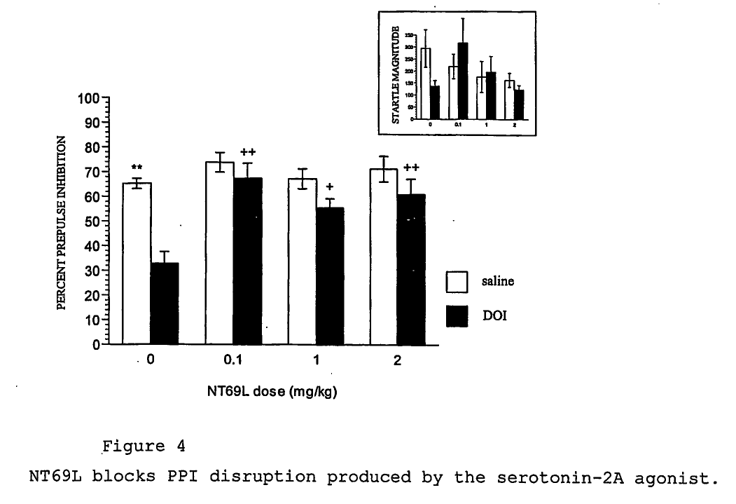

This disclosure demonstrates, that neurotensin agonists also inhibit neural function that is produced by activation of serotonin-2 receptors, an effect similar to second-generation antipsychotics and similar to several current drugs developed and approved to treat depression and anxiety. This is a property of these compounds heretofore unrecognized and produced by a different mechanism since NT and NT agonists do not bind directly to serotonin-2A receptors. When administered to rats, both PD149163 and NT69L, neurotensin agonists produced by modifying the 8-13 fragment of neurotensin to enhance entry in the brain after systemic administration, reversed PPI disruption produced by the serotonin-2A agonist, DOI (see, e.g., FIG. 4, see also Feifel et al., Neuropsychopharmacology, 28(4):651-3 (2003)). All drugs that produce this effect are known to inhibit neuronal transmission that is mediated by serotonin-2A receptors.

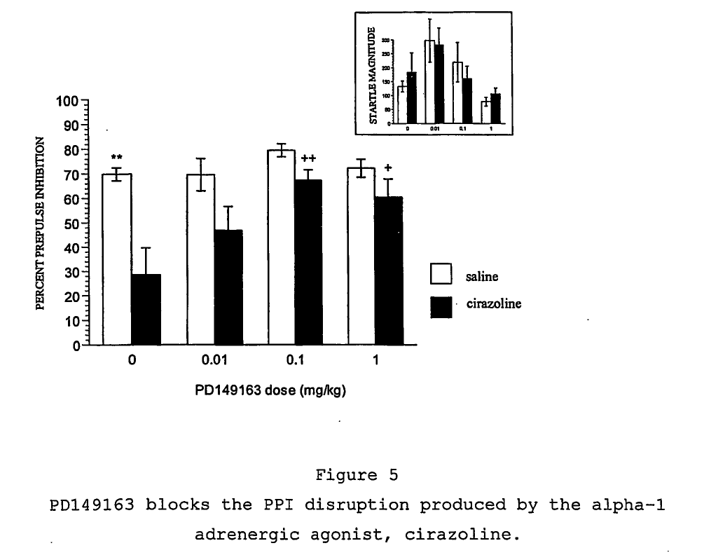

This disclosure demonstrates, that neurotensin agonists also inhibit neural transmission that is produced by activation of alpha-1 adrenergic receptors, a pharmacological effect produced by many antipsychotics and which appears to contribute their clinical efficacy. This is also a property of heretofore unrecognized to be associated with neurotensin agonists. When administered to rats, both PD149163 and NT69L, reversed PPI disruption produced by the alpha-1 adrenergic agonist, cirazoline (see, e.g., FIGS. 5, 6, 8, and 10).

Without wishing to be bound by a particular mechanistic description, since neither PD149163 or NT69L bind to serotonin-2A or alpha-1 adrenergic receptors, the ability of these neurotensin agonists is probably produced by their binding to neurotensin receptors in the brain, which in turn modulates neural transmission in circuits mediated by serotonin-2A and alpha-1 adrenergic receptors.

This disclosure demonstrates also that in genetic animal models of neuropychiatric diseases associated with reduced PPI, neurotensin agonists produce an increase in PPI, that is more robust than clozapine, the antipsycotic drug considered to have the greatest therapeutic efficacy among all currently available antipsychotic drugs. When administered to BB and BN rats PD149163 significantly increased PPI after a single dose. Haloperidol, the prototypical first-generation antipsychotic, did not alter PPI in BB or BN rats. Clozapine, the prototypical second-generation antipsychotic, and the drug with the strongest record of therapeutic efficacy, increased PPI at certain doses in BB but not BN rats. PD149163 produced a greater increase in PPI in BB and BN rats than clozapine.

This disclosure demonstrates also that a neurotensin agonist can increase PPI beyond normal levels. When administered to wild type Long Evans rats that have normal PPI, high doses of PD149163 increased PPI (see, e.g., FIG. 9).

These results suggested that NT agonists including, but not limited to, PD149163 produce antipsychotic-like preclinical effects by mechanisms other than inhibition of dopamine transmission alone and that the preclinical profile of, for example, PD149163 is more similar to the atypical than typical antipsychotics, but that the profile is in other ways suggestive of greater clinical efficacy than first- or second-generation antipsychotics since, for example, PD149163 had stronger effects than clozapine or haloperidol in BB and BN rats.

The disclosure demonstrates that neurotensin agonists are useful in reversing PPI deficits produced by a wide variety of pharmacological interventions and genetic causes and thus are useful in treating a wide array of neuropsychiatric disorders in which sensorimotor gating is disrupted.

Furthermore, the disclosure provides that neurotensin agonists may facilitate PPI even among animals that do not have a deficient PPI, thus demonstrating that neurotensin agonists may improve normal cognitive functions in subject that have a normal PPI.

Accordingly, the disclosure provides methods of treating a neuropsychiatric disorder by administering and effective amount of an NT agonist to a subject such that the neuropsychiatric disorder is modulated. In one aspect the administration inhibits one or more symptoms of a neuropsychiatric disorder. As used herein, the term “modulate” or “modulation” shall have its usual meaning, and encompasses the meanings of the words “enhance,” “inhibit,” and “mimic.” “Modulation” of activity may be either an increase or a decrease in activity. The term “effective dose” as used herein refers to any amount of compound that induces the particular described response without inducing significant toxicity. For example, an effective dose of an NT agonist (e.g., NT69L or PD149163) for schizophrenia can be that amount needed to cause the mammal (e.g., human) to exhibit behavior consistent with a normal psyche without significant toxicity. In addition, an effective dose of a particular compound administered to a mammal can be adjusted according to the mammal's response and desired outcomes. Significant toxicity can vary for each particular subject or patient and depends on multiple factors including, without limitation, the patient's degree of illness, age, weight, size, and gender. In addition, an effective amount is an amount that can be determined by one of skill in the art based on data from studies using methods of analysis such as those disclosed herein. Such data may include, but not be limited to, results from IC₅₀ determinations, as discussed herein or carried out in the art.

In another aspect, administration of NT agonist can assist in an improved cognitive behavior in normal subjects (e.g., subject that do not have a neuropsychiatric disorder). Such improved cognitive behavior can include, without limitation, improved memory, attention, sensory perception, learning ability, and the like.

For example, as demonstrated herein, the NT agonist, NT69L, and PD149163 blocked DOI-induced PPI deficits. The data provide support for the notion that NT agonists inhibit serotonin-2A mediated modulation of neurotransmission. In this regard, all drugs reported to antagonize PPI deficits produced by DOI exhibit strong serotonin-2A antagonism, such as the atypical antipsychotics, clozapine and risperidone, and the selective serotonin-2A antagonists, MDL100907 and ketanserin (Geyer et al. supra). In contrast, drugs that do not have high affinity for serotonin-2A receptors, such as haloperidol, the selective DOPAMINE-2 antagonist raclopride, the 5HT2C antagonist SDZ SER-082, and the beta adrenergic/5HT1 antagonist propanolol, do not block DOI-induced PPI deficits (Geyer et al, supra).

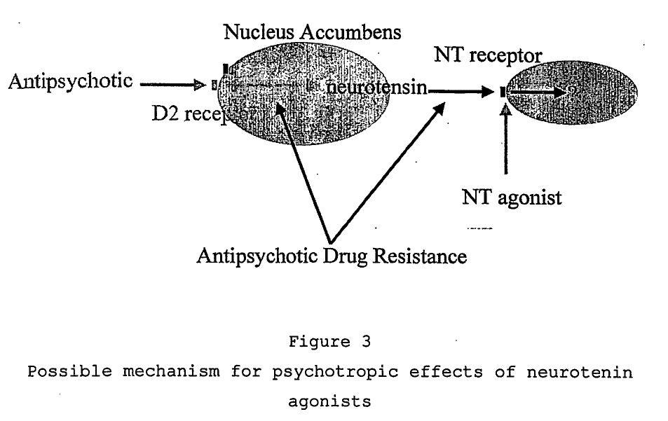

Although NT agonists and atypical antipsychotics exhibit similar effects on DOI-induced PPI deficits, the mechanism of action is different. Atypical antipsychotics act as serotonin-2A receptor antagonists. In contrast, NT agonists (such as those provided herein) do not act as strong serotonin-2A antagonists since they exhibit low affinity for this receptor. Therefore, the effects of NT agonists on serotonin-2A mediated neurotransmission are mediated downstream to the serotonin-2A receptor. An exemplary pathway and the role of NT analogues are depicted in FIG. 3.

NT and serotonin systems interact in the brain on many levels. For example, there are NT-containing cell bodies in most rostral raphe nuclei, and NT-containing fibers and terminals in all raphe nuclei. NT induces a concentration-dependent increase in firing rate of a subpopulation of serotonergic neurons in the ventral part of the dorsal raphe nucleus, which can be blocked by SR48692, a NT receptor antagonist. Based upon this it would be expected that NT agonists would facilitate rather than inhibit serotonin-2A function.

This disclosure demonstrates that NT agonists including, for example, PD149163 and NT69L, reverse cirazoline-induced PPI deficits, which suggest that inhibition of alpha-1 modulation of neural function is also a property of NT agonists. For example, NT69L has minimal affinity for monoamine receptors and thus the effects of this compound on alpha-1 receptor mechanisms are most likely downstream. Both NT and alpha-1 receptors have similar signal transduction pathways via inositol triphosphate and therefore NT agonists can modulate via this common signal transduction pathway, downstream transduction effects through alpha-1 receptors. Evidence exists that inhibition of alpha-1 function can contribute to the therapeutic effects of antipsychotic drugs (Geyer et al. supra). For example, many typical and atypical antipsychotic drugs exhibit high affinity for alpha-1 receptors. Alpha-1 antagonists do not have antipsychotic efficacy on their own, but this pharmacological effect can augment the therapeutic efficacy as well as the preclinical effects that are predictive of therapeutic efficacy of antipsychotic drugs. For example, the alpha-1 noradrenergic antagonist, prazosin, enhanced changes in the conditioned avoidance response produced by the dopamine-2 receptor antagonist, raclopride. These effects may occur by modulation of dopamine release in the nucleus accumbens.

Thus, NT agonists block PPI deficits produced by dopamine, serotonin-2A, alpha-1 adrenergic agonists and non-competitive glutamate antagonists. In addition, NT agonists exhibit low affinity for monoamine and glutamate receptors. NT agonists produce their effect on PPI deficits indirectly by modulating neural circuitry that inhibits a common output of the circuitry affected by these neurotransmitter systems. Thus, the disclosure provides methods of inhibiting serotonin-2A and/or alpha-1 receptor mediated modulation of neural function comprising administering to a subject an effective amount of a neurotensin agonist.

NT agonists or their salts can be formulated for deliver by admixture with pharmaceutically acceptable non-toxic excipients or carriers. Mention may be made, as examples of pharmaceutically acceptable salts, of the addition salts with inorganic or organic acids (such as acetate, trifluoroacetate, propionate, succinate, benzoate, fumarate, maleate, oxalate, methanesulphonate, isethionate, theophyllinacetate, salicylate, methylenebis-β-oxynaphthoate, hydrochloride, sulphate, nitrate and phosphate), the salts with alkali metals (sodium, potassium or lithium) or with alkaline-earth metals (calcium or magnesium), the ammonium salt or the salts of nitrogenous bases (ethanolamine, trimethylamine, methylamine, piperidine, benzylamine, N-benzyl-.alpha.-phenethylamine, choline, arginine, leucine, lysine or N-methylglucamine).

The disclosure provides pharmaceutical compositions of NT agonists or their salts. The NT agonist or their physiologically acceptable salts or solvates, may be formulated for administration for injection, or for oral, topical, nasal, inhalation, insufflation (either through the mouth or the nose) buccal, parenteral, rectal administration or other forms of administration. The disclosure provides pharmaceutical compositions comprising effective amounts of an NT agonist together with pharmaceutically acceptable diluents, preservatives, solubilizers, emulsifiers, adjuvants, excipients and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimerosal, benzyl alcohol) and bulking substances (e.g., lactose, mannitol).

The compositions may also be incorporated into particulate preparations of polymeric compounds such as polylactic acid, polyglycolic acid, and the like or liposomes. Hyaluronic acid may also be used. Biocompatible absorbable polymers may be selected from the group consisting of aliphatic polyesters, copolymers and blends, which include, but are not limited to, homopolymers and copolymers of lactide (which include D-, L-, lactic acid and D-, L- and meso lactide), glycolide (including glycolic acid), epsilon-caprolactone, p-dioxanone (1,4-dioxan-2-one), alkyl substituted derivatives of p-dioxanone (i.e., 6,6-dimethyl-1,4-dioxan-2-one), triethylene carbonate (1,3-dioxan-2-one), alkyl substituted derivatives of 1,3-dioxanone, delta-valerolactone, beta-butyrolactone, gamma-butyrolactone, epsilon-decala tone, hydroxybutyrate, hydroxyvalerate, 1,4-dioxepan-2-one and its dimer 1,5,8,12-tetraoxacyclotetradecane-7,14 dione, 1,5-dioxepan-2-one, and polymer blends thereof.

Such compositions may influence physical state, stability, rate of in vivo release, and rate of in vivo clearance. See, e.g., Remington s Pharmaceutical Sciences, 18th ed., (1990, Mack Publishing Co., Easton, Pa. 18042) pages 1435-1712). The compositions may be prepared in liquid form, or be in dried powder, such as lyophilized form.

Contemplated for use herein are oral solid dosage forms, which are disclosed generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets or pellets. Also, liposomal or proteinoid encapsulation may be used to formulate compositions. Liposomal encapsulation may be used and the liposomes may be derivatized with various polymers. A description of possible solid dosage forms for the therapeutic is given by Marshall, K. In: Modern Pharmaceutics Edited by G. S. Banker and C. T. Rhodes Chapter 10, 1979). In general, the formulation will include an NT agonist and inert ingredients (which allow for protection against the stomach environment and release of the biologically active material in the intestine).

To ensure full gastric resistance a coating impermeable to at least pH 5.0 is useful. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L3OD, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings that make the tablet easier to swallow. Capsules may consist of a hard shell (such as gelatin) for delivery of dry therapeutic, i.e., powder; for liquid forms, a soft gelatin shell may be used. The shell material of cachets may be thick starch or other edible paper. For pills, lozenges, molded tablets or tablet triturates, moist massing techniques can be used.

The therapeutic can be included in the formulation as fine multi-particulates in the form of granules or pellets. The formulation of the material for capsule administration can also be as a powder, lightly compressed plugs or even as tablets. The therapeutic can also be prepared by compression.

Colorants and flavoring agents may all be included. For example, the peptide (or derivative) may be formulated (such as by liposome or microsphere encapsulation) and then further contained within an edible product, such as a refrigerated beverage containing colorants and flavoring agents.

One may dilute or increase the volume of the therapeutic with an inert material or filler. These diluents or fillers can include carbohydrates, especially mannitol, a-lactose, anhydrous lactose, cellulose (e.g., microcrystalline cellulose), sucrose, calcium hydrogen phosphate modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include, but are not limited to, starch (e.g., potato starch or the commercial disintegrant based on starch, Explotab). Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch (e.g., pregelatinised maize starch) and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) can both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes, talc and silica. Soluble lubricants may also be used such as sodium lauryl sulfate, magnesium lauryl sulfate, polyethylene glycol of various molecular weights, Carbowax 4000 and 6000.

Glidants that can improve the flow properties of the drug during formulation and to aid rearrangement during compression can be added. The glidants can include starch, talc, pyrogenic silica and hydrated silicoaluminate.

To aid dissolution of the therapeutic into the aqueous environment a surfactant can be added as a wetting agent. Surfactants may include anionic detergents such as sodium lauryl sulfate, dioctyl sodium sulfosuccinate and dioctyl sodium sulfonate. Cationic detergents can be used and can include benzalkonium chloride or benzethomium chloride. The list of potential non-ionic detergents that can be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty cid ester, methyl cellulose and carboxymethyl cellulose. These surfactants can be present n the formulation of the protein or derivative either alone or as a mixture in different ratios.

Additives that potentially enhance uptake of the agent are, for example, the fatty acids oleic acid, linoleic acid and linolenic acid.

Controlled release oral formulation may be desirable. The agent can be incorporated into an inert matrix that permits release by either diffusion or leaching mechanisms, e.g., gums. Slowly degenerating matrices may also be incorporated into the formulation. Some enteric coatings also have a delayed release effect.

Other coatings may be used for the formulation. These include a variety of sugars that can be applied in a coating pan. The therapeutic agent can also be given in a film coated tablet and the materials used in this instance are divided into two groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials can be used to provide the optimum film coating. Film coating may be carried out in a pan-coater or in a fluidized bed or by compression coating.

Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Nasal delivery of an NT agonist is also contemplated. Nasal delivery allows the passage of the protein to the blood stream and into the brain directly after administering the therapeutic product to the nose, without the necessity for deposition of the product in the lung. Formulations for nasal delivery include those with dextran or cyclodextran.

For administration by inhalation, the NT agonist is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The NT agonists may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations disclosed previously, the NT agonists may also be formulated as a depot preparation. Such long acting formulations may be administered by implantation (for example subcutaneously or intramuscularly) or by intramuscular injection. Thus, for example, the NT agonists may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Toxicity and therapeutic efficacy of such NT agonists can be determined by standard pharmaceutical procedures in cell cultures or experimental animal/animal models (such as those described herein), e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED₅₀ with little or no toxicity. The dosage may vary within this range depending upon the dosage form employed and the route of administration utilized. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

An NT agonist and components of a therapeutic composition may be introduced parenterally, topically, or transmucosally, e.g., orally, nasally, or rectally, or transdermally. Parenteral administration includes, for example, intravenous injection, intra-arteriole, intramuscular, intradermal, subcutaneous, intraperitoneal, intraventricular, and intracranial administration.

Because many NT agonist are capable of crossing the blood brain barrier NT agonists permit oral, parenteral or intravenous administration. Alternatively, the agent can be modified or otherwise altered so that it can cross or be transported across the blood brain barrier. Many strategies known in the art are available for molecules crossing the blood-brain barrier, including but not limited to, increasing the hydrophobic nature of a molecule; introducing the molecule as a conjugate to a carrier, such as transferring, targeted to a receptor in the blood-brain barrier, or to docosahexaenoic acid and the like.

In another embodiment, an NT agonist may be administered by surgical intervention including a procedure of drilling a small hole in the skull to administer the agent.

In another embodiment, the molecule can be administered intracranially or intraventricularly. In another embodiment, osmotic disruption of the blood-brain barrier can be used to effect delivery of agent to the brain (Nilayer et al., Proc. Natl. Acad. Sci. USA 92:9829-9833 (1995)). In yet another embodiment, an agent can be administered in a liposome targeted to the blood-brain barrier. Administration of pharmaceutical agents in liposomes is known (see Langer, Science 249:1527-1533 (1990); Treat et al., in Liposomes in the Therapy of infectious Disease and Cancer, Lopez-Berestein and Fidler (eds.), Liss, New York, pp. pp. 317-327 and 353-365 (1989).

Although some predictions have been made concerning the ability of molecules to pass through the blood-brain barrier, the rate and extent of entry of an NT agonist or a formulation comprising an NT agonist into the brain are generally considered to be determined by partition coefficient, ionization constant(s), and molecular size.

In another embodiment, a therapeutic formulation comprising an NT agonist can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss: New York, pp. 317-327 and 353-365 (1989)).

In another embodiment, a therapeutic formulation comprising an NT agonist can be delivered in a controlled release system. For example, the NT agonist may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

In addition, any of the materials described herein can be administered to any part of the mammal's body including, without limitation, brain, spinal fluid, blood stream, lungs, nasal cavity, intestines, stomach, muscle tissues, skin, peritoneal cavity, and the like. Thus, an NT agonist (e.g., a neurotensin analog) can be administered by intravenous, intraperitoneal, intramuscular, subcutaneous, extracranial, intrathecal, and intradermal injection, by oral administration, by inhalation, or by gradual perfusion over time. For example, an aerosol preparation can be given to a mammal by inhalation. It is noted that the duration of treatment with the materials described herein can be any length of time from as short as one day to as long as a lifetime (e.g., many years). For example, an formulation comprising an NT agonist can be administered once (or twice, three times, etc.) daily, weekly, monthly, or yearly.

Preparations for administration can include sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water as well as alcohol, saline, and buffered solutions. Preservatives, flavorings, and other additives such as, for example, antimicrobials, anti-oxidants, chelating agents, inert gases, and the like may also be present. In another aspect, the disclosure provides animal models useful for identifying agents that modulate PPI and thus have clinical and therapeutic potential as treatments for psychotic disorder. Preclinical paradigms that can differentiate atypical from typical clinical profiles are needed since advances in the therapeutic field make it no longer desirable to develop compounds with first generation clinical profiles. Since it is established that atypical antipsychotics can produce desirable clinical effects not associated with typical antipsychotics, it is reasonable to assume that there exist preclinical paradigms, which model this atypical antipsychotic clinical advantage. Thus, whereas sensitivity to haloperidol was once considered the “litmus test” for validating preclinical models of antipsychotic potential, it is now desirable to develop preclinical models that are preferentially sensitive to atypical antipsychotics over typical antipsychotics such as haloperidol. Indeed, several preclinical paradigms have been proposed to be useful to identify putative antipsychotics of atypical category. Typically in these paradigms second generation antipsychotics have a spectrum of effects that is distinct or broader than typical antipsychotics. Examples of these effects include antagonism of PPI disruption produced by NMDA antagonists (Geyer et al., supra) and induction of a distinct regional pattern of immediate early gene expression.

The effects of PD149163 on PPI disruption produced by the serotonin-2A agonist, 1-(2,5-dimethoxy-4-iodophenyl)-2-aminopropane hydrochloride (DOI) was investigated. The disclosure demonstrates that PD149163 blocked the effect of DOI on PPI. This finding is the first suggestion that NT agonists could inhibit serotonin-2A function. This finding has clinical relevance since inhibition of serotonin-2A is considered an important pharmacological mechanism of antipsychotics, in addition to dopamine inhibition. Other NT agonist such as NT69L showed similar results on DOI-induced PPI deficits suggesting that as neurotenin agonists can inhibit neural transmission mediated by serotonin-2A receptors, a mechanism which has proven to be associated with improvement in a broad array of neuropsychiatric symptoms. With out wishing to be bound by the mechanistic effects of NT agonists, NT agonists may inhibit alpha-1 modulation of neurotransmission, since PD149163 and NT69L blocked PPI disruption by the alpha-1 adrenergic receptor agonist cirazoline. This is of clinical relevance since inhibition of alpha-1 neurotransmission is thought to enhance the antipsychotic effects of many antipsychotic drugs including clozapine.

NT agonists, e.g., NT69L and PD149163, blocked PPI deficits produced by DOI and cirazoline and that NT agonists possess the ability to inhibit serotonin-2A and alpha-1 receptor mediated modulation of neural function. These were previously unknown effects of NT agonists. Since evidence exists that inhibition of serotonin-2A and alpha-1 adrenergic function augment dopamine-2 receptor inhibition in regard to antipsychotic efficacy, these findings further support the possibility that NT agonists can be developed as useful antipsychotic drugs. In addition, these results also demonstrate that the putative antipsychotic properties of NT agonists extend beyond the effects of these compounds on dopaminergic neurotransmission. Furthermore, since drugs that are able to inhibit serotonin-2A neurotransmission have been useful in treating a broad array of non-psychotic neuropsychiatric disorders and symptoms, including but not limited to, bipolar mania, bipolar affective disorder, depression, anxiety, autism and obsessive-compulsive disorder either as monotherapy or in conjunction with other psychotropic drugs, neurotenin agonist can be useful as monotherapy or as co-therapy with other psychotropic drugs to treat this neuropsychiatric diseases and symptoms.

Male or female homozygous Brattleboro rats are commercially available (Harlan Sprague Dawley, Inc., Indianapolis, Ind.). A test compound or reference agent is given at an oral dose of 1 to 10 mg/kg in a volume of 10 ml/kg. The vehicle used can be any pharmaceutically acceptable carrier including saline, sterile water, DMSO, and the like.

Untreated (vehicle) BB rats exhibited reduced PPI compared to untreated LE rats in each of the three experiments. Acute administration of haloperidol did not affect PPI in either LE or BB rats.

Both higher and lower doses in addition to the 0.5 mg/kg dose of haloperidol did not show a significant effect on PPI, it can be reasonably conclude that the PPI deficit in BB rats is not affected by acute administration of haloperidol.

It is significant that clozapine was able to reverse PPI deficits in BB rats. This reversal was dose-dependent with the 10 and 15 mg/kg dose exhibiting the greatest efficacy. It is not likely that clozapine's ability to acutely reverse PPI deficits in BB rats is due to a non-specific pharmacological effect not associated with its therapeutic mechanism, sedation for example. Whereas non-specific effects such as sedation typically reduce normal behavior, for example, locomotor activity, and can thus appear similar to the specific pharmacological effects of antipsychotics, restoration of deficient process, particularly an information processing deficit such as PPI is unlikely to be produced by a non-specific effect. Consistent with this notion Depoortere et al. (1997) concluded that clozapine's enhancement of PPI was not likely due to its sedating properties since sedating psychotropic drugs that do not have antipsychotic properties, for example, diazepam, decrease rather than facilitate PPI (Depoortere et al., 1997).

While there is some debate as to what pharmacological properties underlie the clinical advantages associated with atypical antipsychotics, there is general agreement that combination of antagonism at both the serotonin-2A and dopamine-2 receptors is vital.

The disclosure demonstrates that PD149163 produced a very distinct reversal of PPI deficits in BB rats. In fact, after treatment with the lowest dose, PPI in BB rats was higher than LE rats. The disclosure also provides evidence that indirect inhibition of dopamine transmission cannot account, by itself, for the antipsychotic-like effects of PD149163, since PD149163 was effective in reversing BB rat PPI deficits, whereas haloperidol, a potent dopamine-2 antagonist, was not. The disclosure also demonstrates that PD149163's effect on BB PPI was more consistent with clozapine, than with haloperidol. The disclosure also establishes the validity of the BB model as predictive screen for atypical antipsychotic drugs. It is noteworthy that PD149163 produced the most robust reversal of PPI deficits in BB rats of the three compounds tested, a finding that is auspicious for the therapeutic potential of drugs that target neurotensin receptors.

NT agonists useful in the methods of the invention can be identified by, for example, by monitoring any of the biological characteristics described herein both before and after administration (e.g., following administration to a BB or BN rat). In addition, an NT agonist that induces a neurotensin response can interact with a neurotensin receptor (e.g., a rat or human neurotensin receptor). The term “interaction” as used herein means that two components specifically bind each other. Typically, any compound that has a binding affinity for a particular compound in the sub-millimolar range (e.g., K_{d}<1 mM) is considered to interact with that particular compound. For example, a ligand that binds a receptor with an affinity less than 1 mM specifically interacts with that receptor. Examples of NT agonists that interact with a NT receptor include, but not limited to, neurotensin, any fragment thereof including neurotensin (e.g., from about residue x to about residuel3 of SEQ ID NO:1) wherein x is any number less than 11, or any modified version of neurotensin or a neurotensin fragment thereof, including but not limited to (Boc-Lys⁹)-neurotenin(9-13)-methyl ester, (Dab⁹)-neurotensin(8-13), (Dab⁹)-neurotensin(9-13), (Lys⁹, Trp¹¹, Glu¹²)-neurotensin(8-13), PD149163, NT1, NT2, NT64D, NT64L, NT65L, NT66D, NT66L, NT67L, NT69L, NT69L′, NT71, NT72, NT73, NT74, NT75, NT76, and NT77. Nonpeptide compounds may be neurotensin agonists including nonpeptide drugs that are antagonists at certain neurotensin receptors and agonists at others including, but not limited to, levocobastine, SR48692, SR142948

### EXAMPLES

### Example 1

Animals: Ninety-six male Sprague Dawley rats (250-300 grams at testing) were obtained from Harlan Laboratories, San Diego. In the experiments of Example 1, all groups contained an n of 8. Animals were housed in groups of two or three in clear plastic chambers in a climate controlled room on a 12:12 hour light/dark cycle (lights on 7:00 a.m.-7:00 p.m.). The rats were handled prior to testing. All testing occurred during the light phase of the rats' circadian illumination schedule and they were allowed free access to food and water for the extent of the study, except during the actual testing. Behavioral testing was performed between 9:00 am and 4:30 pm beginning 7 days after arrival. All studies described in this publication were carried out in accordance with the “Principles of laboratory and animal care” as described in the Guide for the Care and Use of Laboratory Animals as adopted and promulgated by the National Institutes of Health.

Drugs: PD149163 (LysΨ(CH2NH)-Lys-Pro-Trp-tLeu-Leu-OEt) was generously made available by the NIMH Chemical Synthesis and Drug Supply Program, and SR1 International, Menlo Park, Calif. NT69L (N-methyl-Arg-Lys-Pro-L-neo-Trp-tLeu-Leu) was developed at Mayo Clinic, Jacksonville, Fla., as described in Tyler-McMahon et al. 2000. DOI and cirazoline hydrochloride were obtained from Sigma Chemicals, St. Louis, Mo. All drugs used were dissolved in saline.

Startle Testing and Test Sessions. Startle testing was performed in four identical startle chambers obtained from San Diego Instruments (San Diego, Calif.). Each chamber consisted of a clear non-restrictive Plexiglass cylinder resting on a Plexiglass platform inside a ventilated and illuminated enclosure housed in a sound-attenuated room. A continuous background noise of 65 dB, as well as the various acoustic stimuli, was produced within each chamber by a high-frequency loudspeaker (Radio Shack Supertweeter, San Diego, Calif.). The whole-body startle response of each animal produced vibrations of the Plexiglass cylinder, which were transduced into analog signals by a piezoelectric unit, mounted underneath the Plexiglass platform (Mansbach et al., 1988). These analog signals were then digitized and stored by an interface unit connected to a microcomputer. Startle amplitude was defined as the degree of motion detected by the piezoelectric unit.

The animals were subjected to a 5-minute acclimation to the 65 dB background noise, which continued throughout the session. The acclimation was followed by a 15 minute PPI test session. Five trial types were presented during the test session: 40 msec 120 dB startle pulses (PULSE-ALONE), startle pulses preceded 100 msecs by 20 msec prepulses of either 4, 8, or 12 dB above background, and NO-STIMULUS trials. All trial types were presented in pseudo-random order separated by an average of 15 seconds. In addition, four PULSE-ALONE trials that were not used in the calculation of PPI values were presented at the beginning and at the end of the test session, to habituate animals to the pulse and in order to provide a measure of habituation across the session.

NT69L versus DOI. Thirty-two drug-naïve rats were administered subcutaneous (SC) injections of the following doses of NT69L, 0 (saline), 0.1, 1, or 2 mg/kg. Thirty minutes later, they were injected SC with either saline or DOI (0.5 mg/kg), a dose known to reliably disrupt PPI. Animals were tested in startle chambers thirty minutes later and PPI measured as described. After one week, animals were tested a second time during which treatment and testing procedures were the same except that rats that previously received DOI received saline and vice versa. The dose of NT69L was kept constant on both test days.

PD149163 versus cirazoline. Thirty-two drug-naïve rats were administered SC injections of the following doses of PD149163, 0 (saline), 0.01, 0.1, or 1 mg/kg. Thirty minutes later, they were injected SC with either saline or cirazoline (0.7 mg/kg), a dose known to reliably disrupt PPI. Animals were tested in startle chambers thirty minutes later and PPI measured as described. After one week, animals were tested a second time during which treatment and testing procedures were the same except that rats that previously received cirazoline received saline and vice versa. The dose of PD149163 was kept constant on both test days.

NT69L versus cirazoline. Thirty-two drug-naïve rats were administered SC injections of the following doses of NT69L, 0 (saline), 0.01, 0.1, or 1 mg/kg. Thirty minutes later, they were injected SC with either saline or cirazoline (0.7 mg/kg). Animals were tested as previously described.

Data and Statistical Analysis. A startle response was recorded for all stimuli presentations. From these data PPI measures and startle magnitude were calculated for each animal. PPI for each animal was calculated as a percentage of the PULSE-ALONE startle magnitude using the following formula: [1-(startle magnitude after prepulse-pulse pair/startle magnitude after PULSE-ALONE) X 100]. PPI data were analyzed using a repeated three factor ANOVA with NT69L and PD149163 dose as a between-subject factor and DOI or cirazoline and prepulse intensity as within-subjects factors.

To determine whether DOI or cirazoline would produce the expected PPI deficits, planned paired t-tests were performed to compare PPI exhibited after treatment with DOI/saline or cirazoline/saline versus the corresponding saline/saline. In addition, to test the prediction that both NT69L and PD149163 would block cirazoline-induced PPI deficits, and NT69L would block DOI-induced PPI deficits, paired t-tests were used to compare saline versus DOI and saline versus cirazoline for each active dose of NT69L and PD149163. Bonferroni corrections were used to correct for multiple pairwise comparisons. Two-tailed Dunnett's post hoc tests were used to compare the groups treated with DOI or cirazoline or saline alone to those also treated with active doses of NT69L/drug or PD149163/drug, respectively.

Analysis of mean startle data was carried out using a two-factor ANOVA with drug treatment as a within factor and NT69L and PD149163 dose as a between-subjects factor. Post-hoc pair-wise comparisons were performed as described for the PPI data.

FIG. 4 (Main) displays the effects of NT69L on baseline and DOI-induced PPI deficits. There was a main effect of DOI as it significantly disrupted PPI (F(1,28)=44.329, P<0.001), and a main effect of NT69L (F(3,28)=5.329, P<0.01) reflected in the reversal of DOI-induced PPI deficits. There was a significant main effect of prepulse intensity on percent PPI, reflected in more intense prepulses producing greater PPI (F(2,56)=104.438, P<0.001). There was a significant DOI X NT69L interaction (F(3,28)=6.555, P<0.01). However, there was neither a significant prepulse X DOI, a prepulse X NT69L, nor a prepulse X DOI X NT69L interaction. Therefore, the PPI data presented in FIG. 4 (Main) are the mean of the PPI values produced by each of the individual prepulse intensities. Rats that received saline/DOI exhibited significantly decreased PPI (P<0.001) compared to rats that received saline/saline. All three active doses of NT69L blocked the PPI deficits produced by DOI. In addition, PPI exhibited by rats that received DOI and 0.1 mg/kg (P<0.001), 1 mg/kg (P<0.05), and 2 mg/kg NT69L (P<0.01) was significantly greater than PPI exhibited by rats that received DOI/saline. There was no significant main effect of NT69L or DOI on startle magnitude, and no significant interactions (FIG. 4 Inset).

FIG. 5 (Main) displays the effects of PD149163 on baseline and cirazoline-induced PPI deficits. There was a main effect of cirazoline as it significantly disrupted PPI (F(1,28)=22.878, P<0.001), and a main effect of PD149163 (F(3,28)=4.604, P=0.01) reflected in the reversal of cirazoline-induced PPI deficits. There was a significant main effect of prepulse intensity on percent PPI, reflected in more intense prepulses producing greater PPI (F(2,56)=29.433, P<0.001). There was not a significant cirazoline X PD149163 interaction. In addition, there was neither a significant prepulse X cirazoline, a prepulse by PD149163, nor a prepulse X cirazoline X PD149163 interaction. Therefore, the PPI data presented in FIG. 5 (Main) are the mean of the PPI values produced by each of the individual prepulse intensities. Rats that received saline/cirazoline exhibited significantly decreased PPI (P<0.01) compared to rats that received saline/saline. The two highest doses of PD149163 reversed PPI deficits produced by cirazoline. In this regard, PPI exhibited by rats that received cirazoline and 0.1 mg/kg PD149163 and cirazoline and 1 mg/kg PD149163 was significantly greater than PPI exhibited by rats that received cirazoline and saline (P<0.01 and P<0.05, respectively).

There was a significant main effect of PD149163 on startle magnitude (F(1,28)=3.650, P<0.05). However, there were no significant differences in startle between the saline/cirazoline group and the three cirazoline groups that received active doses of PD149163. There were no other significant main or interaction effects on startle magnitude (FIG. 5 Inset).

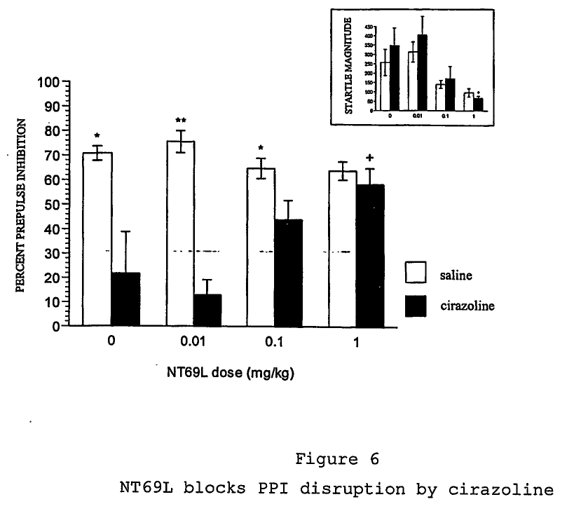

FIG. 6 (Main) displays the effects of NT69L on baseline and cirazoline-induced PPI deficits. There was a significant main effect of cirazoline as it significantly disrupted PPI (F(1,28)=49.822, P<0.001), but no main effect of NT69L. There was a significant main effect of prepulse intensity on percent PPI, reflected in more intense prepulses producing greater PPI (F(2,56)=19.125, P<0.001). There was a significant cirazoline X NT69L interaction (F(3,28)=7.088, P=0.001). There was not a significant prepulse X cirazoline, prepulse X NT69L, or a prepulse X cirazoline X NT69L interaction. Therefore, the PPI data presented in FIG. 6 (Main) are the mean of the PPI values produced by each of the individual prepulse intensities. Rats that received saline/cirazoline exhibited significantly decreased PPI (P<0.05) compared to rats that received saline/saline, and the NT69L dose of 1 mg/kg blocked the PPI deficits produced by cirazoline.

There was a significant main effect of NT69L on startle magnitude (FIG. 6 Inset) (F(3,28)=6.246, P<0.01). Startle exhibited by rats that received cirazoline and 1 mg/kg NT69L was significantly lower than startle exhibited by rats that received cirazoline and saline (P<0.05). There were no significant differences in startle between the saline and the cirazoline group at all active NT69L doses. There were no other significant main or interaction effects.

### Example 2

One hundred forty seven male BB rats and one hundred forty nine LE rats (170-350 grams at testing, Harlan Laboratories, San Diego) were housed in groups of two or three in clear plastic chambers in a climate controlled room under a 12 h/12 h light/dark schedule (lights on/off—7:00 A.M/7:00 P.M). They were allowed free access to food and water for the extent of the study. Behavioral testing was performed 7 days after arrival, during the light phase of the rats' circadian illumination schedule as startle magnitude, PPI and drug effects on PPI are stable across the circadian cycle. The rats were tested in startle chambers to characterize their baseline PPI and startle. Animals were assigned, based on their baseline PPI, to one of four groups matched so as to achieve comparable average PPI across groups. Drug treatment began three days after baseline testing.

In one experiment 50 LE rats and 50 BB rats were administered subcutaneous (SC) injections of either 0 (vehicle), 0.1, 0.5, or 1 mg/kg of haloperidol (UCSD Medical Center, San Diego, Calif.). In another experiment 51 Long Evans rats and 49 Brattleboro rats were administered SC injection of either 0 (vehicle), 5, 10, or 15 mg/kg clozapine (Sigma Chemicals, St. Louis, Mo.). In a third study 43 Long Evans rats and 33 Brattleboro rats were administered SC injection of either 0 (vehicle), 0.5, 1, or 2 mg/kg PD149163. Vehicle for haloperidol was distilled water and the volume injected was 1 ml/kg. Vehicle for clozapine and PD149163 was 0.1 N HCl and half volume 0.9% saline brought to pH 5-6 with a few drops of 1 N NaOH. The volume injected for clozapine was 1.5 ml/kg and for PD149163 was 1 ml/kg. Each treatment group had a minimum of 8 rats.

Animals were tested in startle chambers (San Diego Instruments, San Diego, Calif.) 20 minutes after drug administration. Once placed in startle chambers each rat had a 5-minute acclimation period. A 65-dB background noise was continuously present throughout the session. The acclimation was followed by a 15 minute PPI test session during which rats were presented with 40 msec 120 dB startle pulses without a prepulse, or pulses preceded 100 msec by a prepulse of either 4, 8 or 12 dB above background. These four types of active stimuli were presented in addition to a neutral (no sound) stimuli condition in pseudorandom order with an average of 15 seconds between stimuli types.

A startle response was recorded for all stimuli presentations. PPI for each animal was calculated as a percentage of the pulse-alone startle magnitude using the formula: [1−(startle magnitude after prepulse-pulse pair/startle magnitude after pulse only]×100. Exploratory analysis of the data was conducted and indicated that PPI deficits in BB rats were consistently more robust in the first half of the startle sessions. Therefore, PPI data from this first block of stimuli were subjected to the further statistical analysis. To compare treatments groups, PPI data was subjected to a three-way ANOVA in which prepulse intensity was a within-subject factor and strain and treatment (i.e., drug dose) were between-subject factors. As expected percent PPI was inversely related to prepulse intensity in all three experiments (main effect of prepulse intensity) and this is a well established relationship. However, there was no significant two or three way interaction of prepulse intensity with any of the drugs tested. Therefore, this term was dropped from the model and the analysis reported is based upon a reduced model examining averaged PPI from all prepulse intensities. Pairwise comparisons were made using a Dunnett's one-tailed test to test the following specific hypotheses:
1. PPI in untreated BB rats is significantly lower than in untreated LE rats.
2. Treatment with the test drugs facilitates PPI in BB rats but not in LE rats.
3. Treatment of BB rats with the test drugs restores their PPI to levels of control LE rats.

Data of the acoustic startle response (ASR) to the startle stimuli not preceded by any prepulse were subjected to analysis using a similar two factor ANOVA.

In a separate study to compare the catalepsy effects of each drug, forty-eight drug-naïve BB rats were given one of the following SC treatments: saline, 1 mg/kg haloperidol, 10 mg/kg clozapine, 15 mg/kg clozapine, 1 mg/kg PD149163 or 2 mg/kg PD149163 (n=8 for all groups). Doses selected were those that produced the greatest effect in the PPI studies. Thirty minutes after SC injections animals were tested in the using a method described by several other authors (Stanley and Glick, 1976; Costall et al., 1978; Wadenberg, 1996). This method involved placing the forepaws of each rat in an extended position over a pencil that was suspended horizontally 9 cm above the lab bench. The time spent in this position before the animal moved or corrected itself was considered a measure of catalepsy.

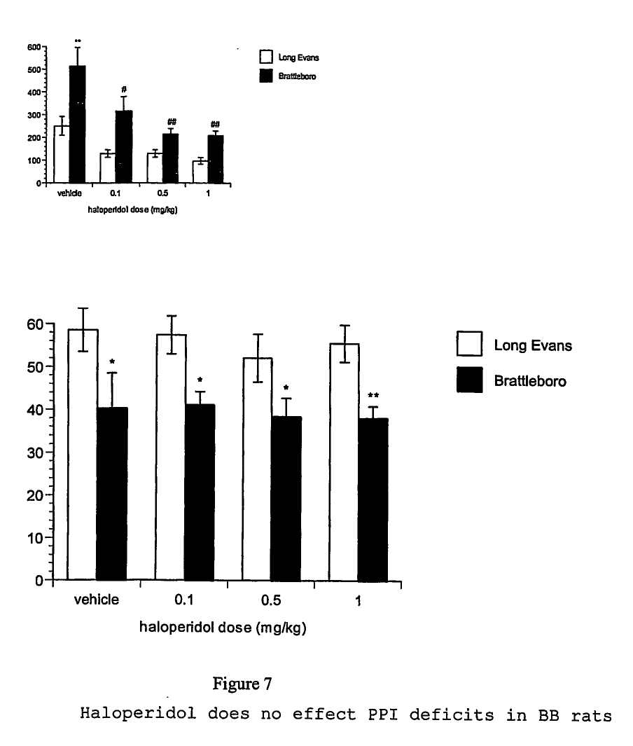

Haloperidol PPI Experiment: FIG. 7A illustrates PPI results in the haloperidol experiment. There was a main effect of strain (F(1,92)=23.3, P<0.001) with LE rats exhibiting higher PPI than BB rats. There was no significant main effect of haloperidol nor was there a significant haloperidol x strain interaction. PPI exhibited by vehicle-treated LE rats was significantly higher (P<0.05) than BB rats for all doses of haloperidol. None of the doses of haloperidol significantly increased PPI in either LE or BB rats.

FIG. 7B illustrates the ASR results in the haloperidol experiment. Analysis of the ASR data indicates that BB rats had significantly higher ASR (F(1,92)=31.6, P<O.OO1) and that haloperidol significantly decreased the ASR in both strains of rats (F(3,92)=13.6, P<0.001). There was not a significant strain x haloperidol interaction.

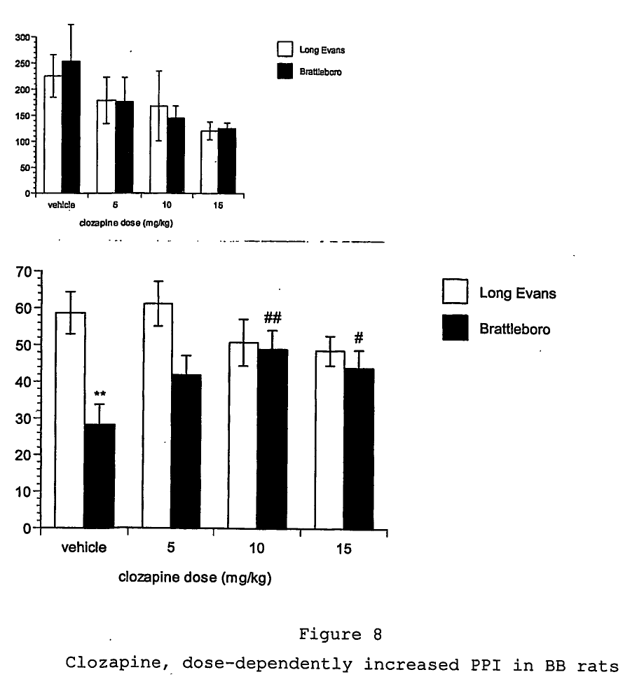

Clozapine PPI Experiment: FIG. 8A illustrates the PPI results in the clozapine experiment. There was a significant main effect of strain (F(1,92)=14.7, P<0.001) and a strain x clozapine interaction (F(3,92)=3.4, P<0.05) but not a main clozapine effect. LE rats treated with vehicle exhibited PPI that was significantly higher than BB rats treated with vehicle (P<0.01) but not significantly different than BB rats treated with any of the doses of clozapine tested. Furthermore, PPI in BB rats treated with vehicle was significantly lower than PPI in BB rats treated with 10 mg/kg (P<0.01) and 15 mg/kg dose (P<0.05) of clozapine. In contrast, no dose of clozapine increased PPI in LE rats.

FIG. 8B illustrates the ASR results in the clozapine experiment. Analysis of the ASR data indicates that there was no significant difference in startle magnitude between BB and LE rats. Clozapine appeared to reduce ASR in both rat strains in a dose-dependent fashion but this effect was slightly above the statistical cut-off for significance (P=0.051). There was no significant clozapine x strain interaction effect.

FIG. 9A illustrates the PPI results in the PD149163 experiment. There was a significant effect of strain ((F(1,68)=6.28, P<0.05), a significant main effect of PD149163, (F(3,68)=4.75, P<0.01), and a significant strain x PD149163 interaction (F(3,68)=4.221, P<0.01). PPI in vehicle-treated LE rats was significantly higher than in vehicle-treated BB rats (P<0.01), but not significantly different from BB rats treated with any of the doses of PD149163. BB rats treated with 1 mg/kg and 3 mg/kg PD149163 had significantly higher PPI (P<0.01 and P<0.05, respectively) than PPI exhibited by vehicle-treated BB rats. In contrast, no dose of PD149163 had a significant effect on PPI in LE rats, although there appeared to be a tendency for PD149163 to dose-dependently increase PPI in LE rats.

FIG. 9B illustrates the ASR data for the PD149163 experiment. There was not a significant difference in the ASR between BB and LE rats but PD149163 significantly decreased the ASR in both strains of rat as evidenced by a main effect of PD149163 (F(3,68)=5.3, P<0.01). There was not a significant strain x PD149163 interaction effect. Each dose of PD149163 significantly reduced (P<0.05) the ASR in BB rats compared to vehicle, whereas the highest dose (3 mg/kg) reduced the ASR in LE rats compared to vehicle.

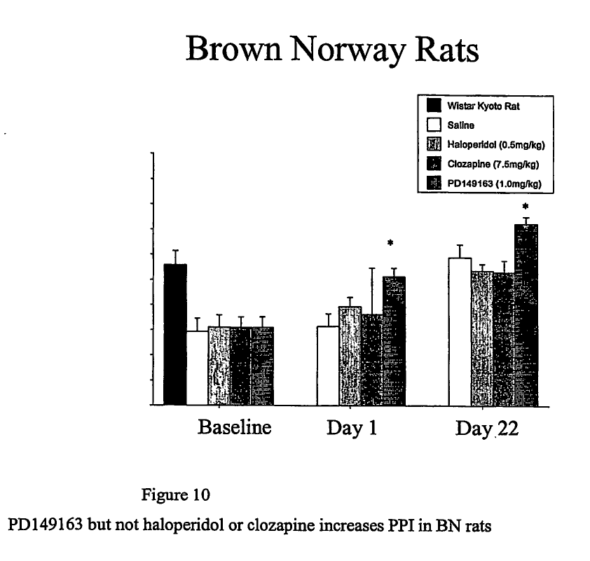

FIG. 10 illustrates the catalepsy findings. There was a main effect of drug treatment (F(5,42)=13.6, P<0.001). Post-hoc comparisons indicated that haloperidol (P<0.001) but no dose of clozapine or PD149163 significantly increased the time rats remained on the bar.

A number of embodiments of the invention have been described. Nevertheless, it will be understood that various modifications may be made without departing from the spirit and scope of the invention. Accordingly, other embodiments are within the scope of the following claims.

1. A method for identifying an agent useful for treating neuropsychiatric disorders comprising:
(a) administering an agent to an animal model having a inherently reduced prepulse inhibition (PPI),
(b) subjecting the animal model to a startle stimulus, and
(c) observing the magnitude of a PPI in the animal model, wherein a change in magnitude of the PPI compared to a control animal is indicative of an agent that is useful for treating neuropsychiatric disorders.

2. The method of claim 1, wherein the animal model is a Brattleboro Rat model or a Brown Norway Rat model.

3. The method of claim 1, wherein the agent is selected from the group consisting of a polypeptide, a peptide, a small molecule, a peptidomimetic, and a polynucleotide.

4. The method of claim 1, wherein the agent is administered by a route selected from the group consisting of parenterally, topically, subcutaneously and transmucosally.

5. The method of claim 1, wherein the startle stimulus is a physical stimulus selected from the group consisting of an auditory stimulus, a visual stimulus, and a nocioceptive stimulus.

6. The method of claim 1, wherein the neuropsychiatric disorder is selected from the group consisting of Schizophrenia, schizoaffective disorder, bipolar disorder, Huntington's disorder, Tourette disorder, Obsessive-Compulsive Disorder, major depression, anxiety and autism.

7. A method for screening a test psychotropic agent for treating a neuropsychiatric disorder comprising:
(a) administering to an animal comprising an inherently reduced pre-pulse inhibition a test psychotropic agent; and
(b) measuring prepulse inhibition in a startle reflex response chamber, wherein an increased prepulse inhibition level indicates a strong clinical potential as a psychotropic drug.

8. The method of claim 7, wherein the animal is a Brown Norway Rat or a Brattleboro Rat.

9. The method of claim 7, wherein the neuropsychiatric disorder is schizophrenia.

10. The method of claim 7, wherein the neuropyschiatric disorder is selected from the group consisting of psychotic depression, postpartum depression, affective disorder, depression, anxiety, schizoaffective disorder, schizophreniform disorder, delusional disorder, brief psychotic disorder, shared psychotic disorder, borderline personality disorder, manic-depressive disorder, obsessive-compulsive disorder, Huntington's Disease, Tourette's syndrome, bipolar disorder, autism and tic disorders.

11-14. (canceled)

15. A method of modulating sensorimotor gating in a subject having inherently reduced pre-pulse inhibition or a disease or disorder causing a reduced pre-pulse inhibition, the method comprising administering to the subject an agent selected from the group consisting of a neurotensin (NT), an NT analog, an NT agonist, a neurotensin mimetic and a combination thereof, in an amount effective to increase prepulse inhibition thereby modulating sensorimotor gating.

16. A method of improving symptoms in a subject comprising administering to a subject an agent selected from the group consisting of a neurotensin (NT), an NT analog, an NT agonist, a neurotensin mimetic and a combination thereof, as monotherapy or in combination with other psychotropic drugs, in an amount effective to increase prepulse inhibition thereby modulating sensorimotor gating.

17. The method of either claim 15 or 16 wherein the agent is selected from the group consisting of neurotensin (from about residue x to about residue 13 of SEQ ID NO:1) wherein x is any number less than 11, or any modified version of neurotensin or a neurotensin fragment thereof selected from the group consisting of (Boc-Lys9)-neurotenin(9-13)-methyl ester, (Dab9)-neurotensin(8-13), (Dab9)-neurotensin(9-13), (Lys9, Trp11, Glu12)-neurotensin(8-13), PD149163, NT1, NT2, NT64D, NT64L, NT65L, NT66D, NT66L, NT67L, NT69L, NT69L′, NT71, NT72, NT73, NT74, NT75, NT76, and NT77; and a compound selected from the group consisting of levocobastine, SR48692, and SR142948.

18. The method of either claim 15 or 16 wherein the agent is administered by a route selected from the group consisting of parenterally, topically, subcutaneously, subdermally, and transmucosally.

19. The method of either claim 15 or 16, wherein the NT agonist is PD149163.

20. The method of either claim 15 or 16, wherein the NT agonist is NT69L.

21. The method of either claim 15 or 16, wherein the subject has a neuropsychiatric disorder.

22. The method of either claim 15 or 16 wherein the subject has a disorder associated with sensorimotor gating abnormalities.

23. The method of claim 21, wherein the neuropsychiatric disorder is selected from the group consisting of schizophrenia, post-traumatic stress disorder, depression, postpartum depression, affective disorder, anxiety, schizoaffective disorder, schizophreniform disorder, delusional disorder, brief psychotic disorder, shared psychotic disorder, borderline personality disorder, manic-depressive disorder, obsessive-compulsive disorder, autism, pervasive developmental disorder Huntington's Disease, Tourette's syndrome, bipolar disorder, and tic disorders.

24. A method of inhibiting serotonin-2A and/or alpha-1 receptor mediated neural function comprising administering to a subject an effective amount of a neurotensin (NT) agonist.

25. The method of claim 24, wherein the NT agonist is selected from the group consisting of neurotensin, any fragment thereof including neurotensin from about residue x to about residue 13 of SEQ ID NO:1) wherein x is any number less than 11, or any modified version of neurotensin or a neurotensin fragment thereof selected from the group consisting of (Boc-Lys9)-neurotenin(9-13)-methyl ester, (Dab9)-neurotensin(8-13), (Dab9)-neurotensin(9-13), (Lys9, Trp11, Glu12)-neurotensin(8-13), PD149163, NT1, NT2, NT64D, NT64L, NT65L, NT66D, NT66L, NT67L, NT69L, NT69L′, NT71, NT72, NT73, NT74, NT75, NT76, and NT77; and a compound selected from the group consisting of levocobastine, SR48692, and SR142948.

26. The method of claim 24, wherein the NT agonist is administered by a route selected from the group consisting of parenterally, subcutaneously, subdermally, topically, and transmucosally.

27. The method of claim 24, wherein the NT agonist is PD149163.

28. The method of claim 24, wherein the NT agonist is NT69L.

29. The method of claim 24, wherein the subject has a neuropsychiatric disorder.

30. The method of claim 29, wherein the neuropsychiatric disorder is selected from the group consisting of schizophrenia, post-traumatic stress disorder, depression, postpartum depression, affective disorder, anxiety, schizoaffective disorder, schizophreniform disorder, delusional disorder, brief psychotic disorder, shared psychotic disorder, borderline personality disorder, manic-depressive disorder, obsessive-compulsive disorder, autism, pervasive developmental disorder Huntington's Disease, Tourette's syndrome, bipolar disorder, and tic disorders.

31. A method of improving cognitive function and/or memory attention in a subject comprising administering to a subject an agent selected from the group consisting of a neurotensin (NT), a neurotensin fragment, an NT analog, an NT agonist, and a combination thereof, in an amount effect to improve cognitive function and/or memory attention compared to a control subject.

32. A method for treating a subject having a neuropsychiatric disorder comprising administering to the subject a pharmaceutically effective dose of an NT agonist or a pharmaceutically acceptable salt thereof.

33. The method of claim 32, wherein the pharmaceutically effective dose is between about 50 and about 300 mg per day, with unit doses in the range of about 10 to about 100 mg.

34. The method of claim 32, wherein the administering is oral, parental, subcutaneous, intranasal, rectal or topical.
